(19) United States
(12) Patent Application Publication
(10) Pub. No.: US 2026/0354762 A1
(43) Pub. Date: Nov. 12, 2026

(54) WEARABLE DEVICE WITH MOTION-BASED TAP INTERACTION AND HAPTIC COMMUNICATION

(71) Applicant: Pulse Mindfulness Inc., Wilmington, DE (US)

(72) Inventors: Johan Matton, Berlin (DE); Elliott Bavarian, Stockholm (SE)

(73) Assignee: Pulse Mindfulness Inc., Wilmington, DE (US)

(21) Appl. No.: 19/671,143

(22) Filed: May 7, 2026

Related U.S. Application Data
(60) Provisional application No. 63/803,431, filed on May 9, 2025.
(60) Provisional application No. 63/803,433, filed on May 9, 2025.
(60) Provisional application No. 63/803,437, filed on May 9, 2025.

(51) Int. Cl. A61B 5/00; A61B 5/11; A61B 5/16

(52) U.S. Cl. CPC A61B 5/6826; A61B 5/002; A61B 5/0022; A61B 5/1123; A61B 5/165; A61B 5/486; A61B 5/7285; A61B 5/7455; A61B 2560/0209

(57) ABSTRACT

A wearable device is disclosed comprising a motion sensor for detecting user-generated tapping gestures, a processor for analyzing and recognizing tapping patterns, a memory for storing pattern data or parameters, a vibration element for providing haptic feedback, and optionally a communication interface for transmitting the patterns to another device. The device allows users to define or respond to pulse sequences through motion and vibration, enabling silent, tactile communication between wearable devices or with mobile systems. The device may operate as a standalone interaction interface or as part of a connected system including a second wearable or smartphone. The device can comprise wearable stress monitoring and management features and functions comprising for example a photoplethysmography (PPG) sensor and a wireless charger. Applications include notification, confirmation, authentication, gesture-based control and stress management.

### CROSS-REFERENCE TO RELATED APPLICATIONS

This application claims priority to U.S. Provisional Patent Application No. 63/803,431, entitled “WIRELESS CHARGER FOR A WEARABLE DEVICE,” filed on May 9, 2025.

This application also claims priority to U.S. Provisional Patent Application No. 63/803,433, entitled “WEARABLE DEVICE WITH MOTION-BASED TAP INTERACTION AND HAPTIC COMMUNICATION,” filed on May 9, 2025.

This application also claims priority to U.S. Provisional Patent Application No. 63/803,437, entitled “SMART RING SYSTEM FOR STRESS MONITORING AND COMMUNICATION,” filed on May 9, 2025. All applications are hereby incorporated by reference in their entirety.

### FIELD OF TECHNOLOGY

The present invention significantly improves upon existing smart ring technology by enabling cloud-based haptic communication, customizable vibration patterns, accelerometer-based interaction, and synchronized multi-ring vibrations. This innovation allows users to stay connected in a tactile and mindful way, even when separated by great distances. The present invention relates generally to wearable technology and physiological monitoring systems. More particularly, it relates to a smart ring system configured to monitor stress levels through physiological sensors and provide interactive feedback via haptic and gesture-based communication.

### BACKGROUND OF THE INVENTION

Wearable devices, such as rings, bracelets, and smartwatches, are becoming increasingly popular due to their convenience and portability. Conventional systems typically focus on passive data collection, such as heart rate or sleep metrics, but offer limited capabilities for real-time feedback or user interaction. Small wearables often lack space for traditional user interface elements, such as touchscreens or physical buttons. This limitation restricts their usability, especially when it comes to inputting commands, confirming actions, or communicating with other devices.

In particular, smart rings are commonly used for notifications, activity tracking, or identification, but their interaction methods are limited. Existing solutions typically rely on external mobile applications, voice commands, or indirect gesture detection through broader hand movement, which can be imprecise or impractical in certain contexts.

Smart rings, in particular, present a challenge due to their compact size and lack of user interface components such as screens or physical buttons. This makes it difficult to provide meaningful, real-time feedback or to receive user input directly through the device. Existing systems may offer alerts via mobile applications or vibration, but they often lack a closed-loop feedback system that evaluates user responses based on physiological changes. Furthermore, discreet and intuitive communication methods between the user and the device—or between multiple users—remain largely undeveloped. These devices typically include rechargeable batteries to ensure portability. Improved designs and techniques for charging wearable devices may be needed and are highly desirable. There is therefore a need for new, intuitive, and space-efficient methods for interacting with small wearable devices. Tapping or touching gestures made by the user, either with another finger or against a surface, represent a natural and discrete input method. However, current technologies do not effectively utilize such input to enable rich interaction or communication between wearables.

In addition, current systems often lack the ability to send tactile, personalized feedback in response to user input or external messages. A robust two-way interaction model based on tapping and haptic feedback has not been fully realized in wearable platforms.

Accordingly, there is a need for a system that can:
detect and interpret tapping gestures on a wearable device,
distinguish between different tapping patterns,
provide haptic feedback,
allow user-defined input patterns,
and support communication between multiple wearable devices or external systems.

The present invention addresses these needs by introducing a wearable device that enables tapping-based interaction and communication using integrated motion sensing and vibration technology.

### SUMMARY OF THE INVENTION

The invention provides a wearable device, such as a ring, that enables intuitive user interaction and communication through physical tapping patterns. The device includes a motion sensor to detect tapping gestures, a processor to analyze and recognize pulse patterns, and a vibration element for haptic feedback. A memory stores these patterns or their parameters, which may be predefined or user-defined. The device may further transmit identified patterns to a second device via wireless communication.

The second device may replicate the pattern through haptic feedback and optionally respond with its own tapping input. The system supports both direct ring-to-ring communication and indirect communication via mobile or cloud services. Additional features include power optimization, gesture variation (e.g., rotation), and inter-ring vibration sensing. The invention enables discreet, touch-based communication and command input in a compact wearable form.

The invention also provides a system comprising a wearable ring device and a mobile device configured for bi-directional communication. The ring includes a PPG sensor for measuring physiological signals such as heart rate and heart rate variability, and a motion sensor for detecting tapping gestures. The mobile device receives data, determines the user's stress level, and activates a vibration module in the ring to provide user feedback based on stress analysis.

Importantly, the invention addresses the inherent user interface limitations of ring-type wearable devices, which typically lack displays or physical buttons. By leveraging motion sensing and haptic feedback, the system enables intuitive, screenless interaction with the user. This allows the ring to provide meaningful guidance and receive confirmation or feedback through simple tapping gestures.

The system further detects user interaction via tapping, evaluates whether the haptic instruction led to a physiological response (e.g., stress reduction), and adapts future feedback accordingly. The invention may also support communication between multiple users via tapping and vibration, enabling a silent and intuitive form of interaction.

The present invention provides a wireless charging apparatus that facilitates easy and accurate alignment of a ring-shaped wearable device onto the charger using magnetic attraction. The system includes magnets positioned beneath the charging surface, configured to pull the ring into the correct charging position and hold it securely in place.

### BRIEF DESCRIPTION OF THE DRAWINGS

FIG. 1 illustrates an example of a system comprising a first user wearing a first ring and a second user wearing a second ring.

FIG. 2 illustrates an example of a charging system comprising a charger and a ring.

FIG. 3 illustrates a top view of the example of a charging system comprising a charger and a ring to be charged, when the ring has been set to the charger

FIG. 4 illustrates a ring comprising the technical elements and modules

FIG. 5 illustrates a charger comprising the technical elements and modules

FIG. 6A illustrates an accelerometer signal when tapping a ring

FIG. 6B illustrates different pulses identified from the accelerometer signal

FIGS. 7A, 7B, and 7C illustrate different pulses and delays identified from the accelerometer signal

FIG. 8A illustrates different vibration signals from the vibration element

FIG. 8B illustrates different vibration signals detected as acceleration signals by the accelerometer inside the ring

FIG. 8C illustrates different pulses identified from the acceleration signals

FIG. 9 illustrates one example of the use case comprising a tapping feature

FIG. 10 illustrates one example of the use case comprising a tapping feature

FIG. 11 illustrates one example of the use case comprising a vibration and tapping feature

FIG. 12 illustrates one example of the use case of two rings comprising a vibration and/or tapping feature

FIG. 13 illustrates a block diagram of the complete system including the ring (104), mobile device (106), and optional web service (108)

FIG. 14 illustrates physiological signals recorded over time, highlighting variations during awake and sleep states

FIG. 15 illustrates a high-level flow of the system operation: sensor data collection, data analysis, stress status determination, and feedback via vibration

FIG. 16 provides a detailed flowchart of stress management logic

FIG. 17 illustrates a top view of the example of a charging system comprising a charger and a ring to be charged, when the ring has been set to the charger

FIG. 18 illustrates a side view of the example of a charging system comprising a charger and a ring to be charged, when the ring has been set to the charger

FIG. 19 a) illustrates a top view of the example of a charging system comprising a charger and a ring to be charged, when the ring with a smaller size has been set to the charger

FIG. 19 b) illustrates a top view of the example of a charging system comprising a charger and a ring to be charged, when the ring with a larger size has been set to the charger

FIG. 20 a) illustrates a top view of the example of a charging system comprising a charger and a ring to be charged, when the ring with a smaller size has been set to the charger

FIG. 20 b) illustrates a top view of the example of a charging system comprising a charger and a ring to be charged, when the ring with a larger size has been set to the charger

FIG. 21 a) illustrates a top view of the example of a charging system comprising a charger and a ring to be charged, when the ring with a smaller size has been set to the charger

FIG. 21 b) illustrates a top view of the example of a charging system comprising a charger and a ring to be charged, when the ring with a larger size has been set to the charger

FIG. 22 illustrates a side view of the example of a charging system comprising a charger and a ring to be charged, when the ring has been set to the charger

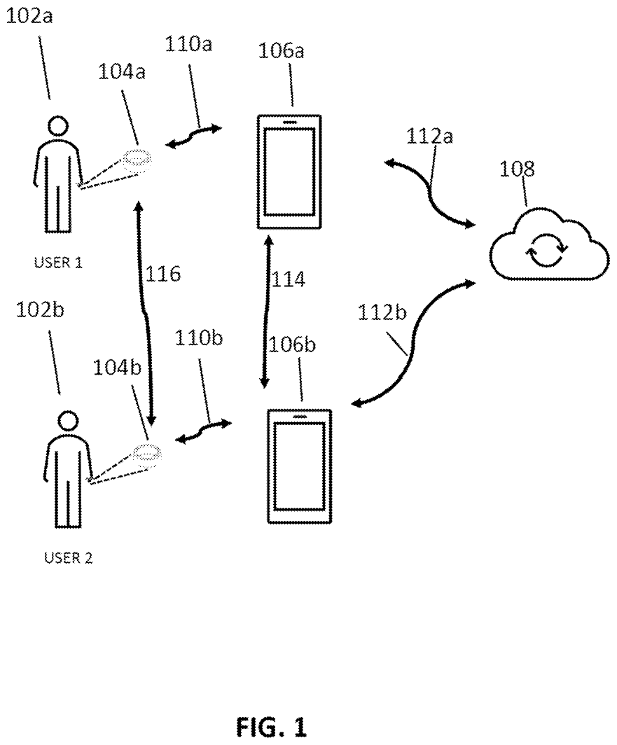

FIG. 1:
102 a and b a user of a ring
104 a and b a ring
106 a and b a mobile device
108 a web service or internet
110 a and b a near field RF communication (Bluetooth, ZigBee, Loran)
112 a and b a mobile cellular network or WiFi (3G, 4G, 5G, 6G, WiFi 2G or 5G)
114 a connection between mobile devices
116 a connection between ring devices

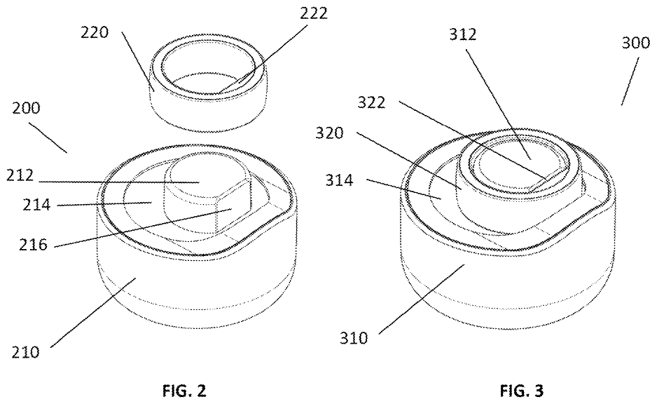

FIG. 2:
200 a charger solution
210 a charger
212 a center part of the charger
214 the surface of the charger on which the ring to be placed
216 the beveled part of the center of the charger
220 a ring
222 the bevel of the ring

FIG. 3:
300 a charger solution
310 a charger
312 a center part of the charger
314 the surface of the charger on which the ring to be placed
320 a ring
322 the bevel of the ring

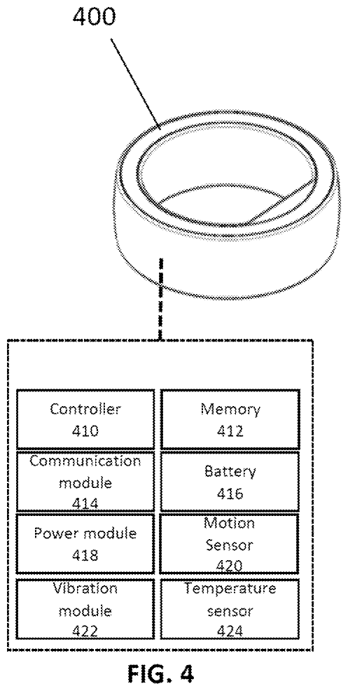

FIG. 4:
400 a ring

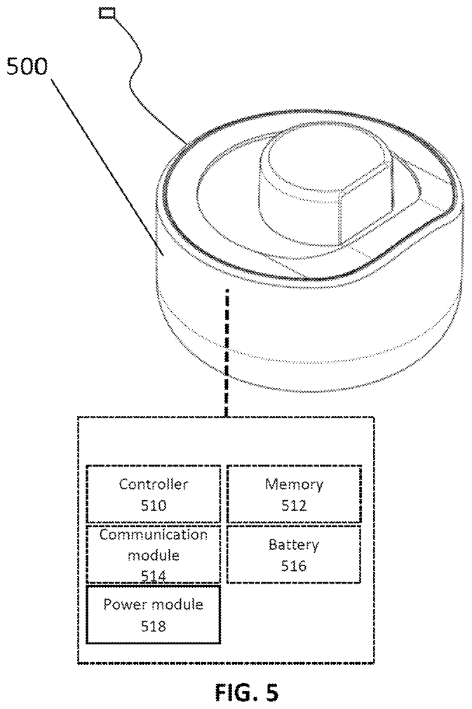

FIG. 5:
500 a charger

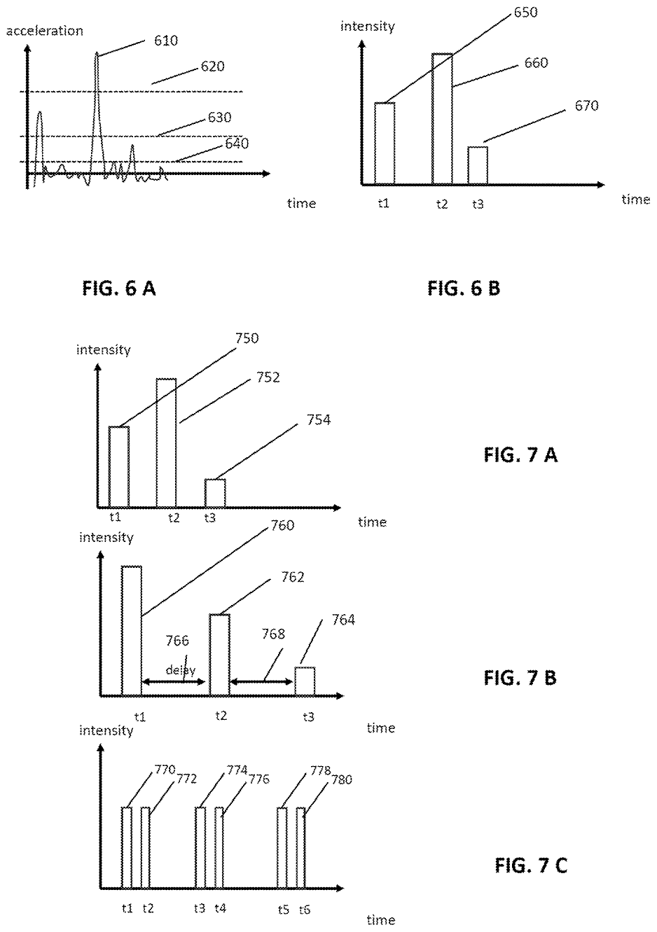

FIG. 6 A and B:
610 an acceleration/motion signal
620 a threshold 1
630 a threshold 2
640 a threshold 3
650, 660, 670 a tapping pulse

FIG. 7 A, B, and C:
750, 752, and 754: vibration signal pulses
760, 762, and 764: vibration signal pulses
766 and 768: a delay between successive pulses
770, 772, 774, 776, 778, and 780: vibration signal pulses

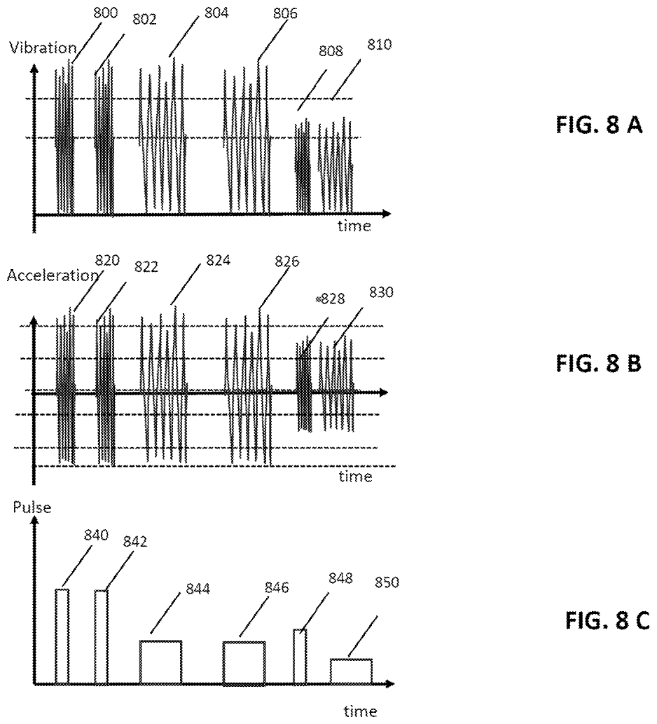

FIG. 8 A, B, and C:
800, 802, 804, 806, 808, and 810 vibration signals
820, 822, 824, 826, 828, and 830 acceleration/motion signals
840, 842, 844, 846, 848, and 850 pulses

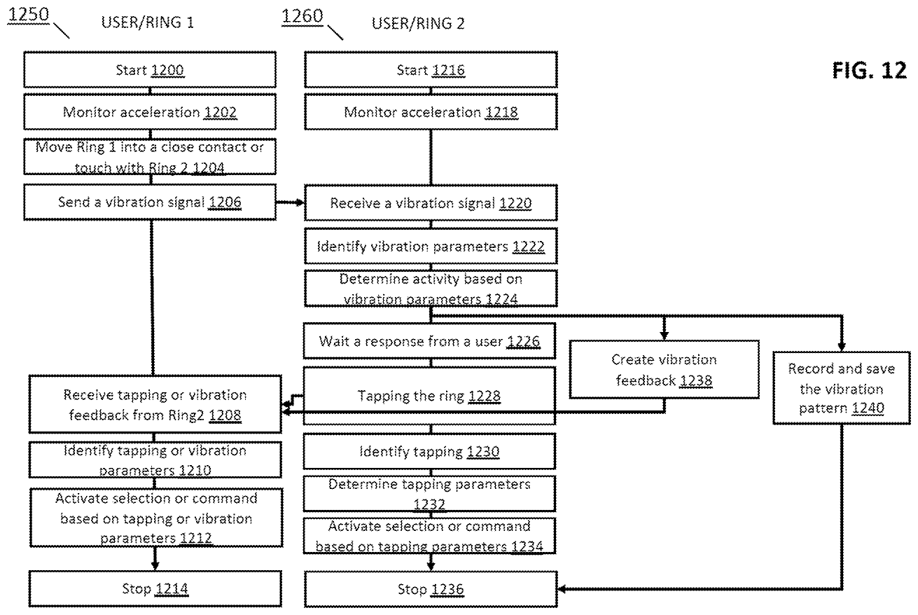

FIG. 12:
1250 An example of the flowchart of the vibration and tapping process in a user device 1
1260 An example of the flowchart of the vibration and tapping process in a user device 2

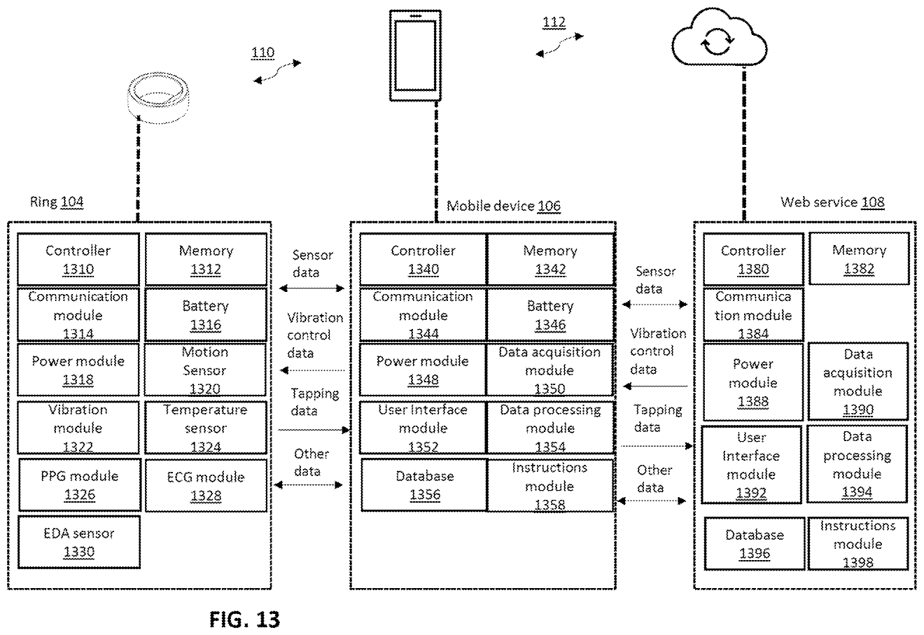

FIG. 13:
104 a ring
106 a mobile device
108 a web service

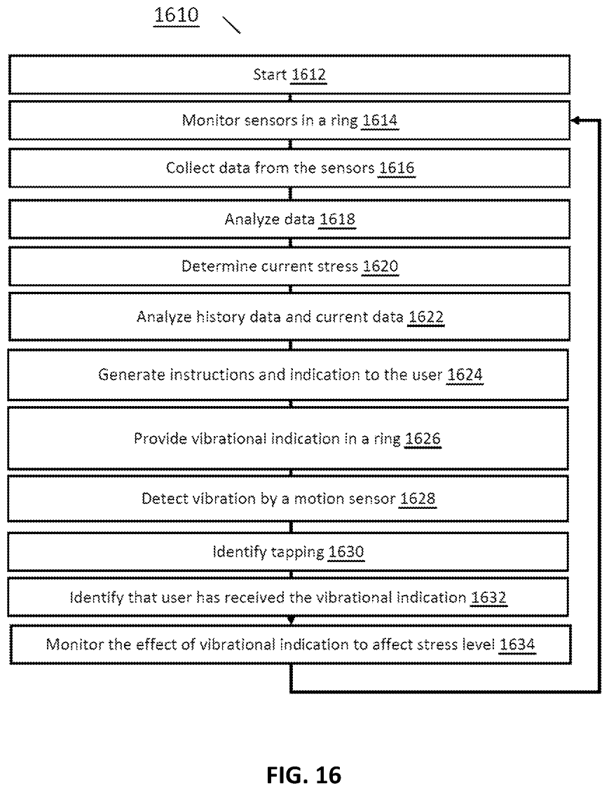

FIG. 16:
1610 a flowchart of a logic

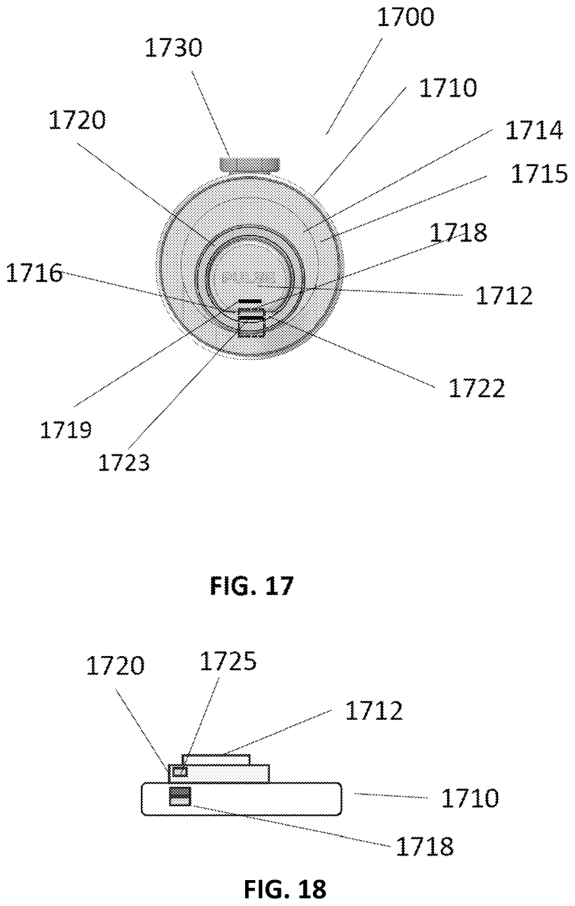

FIG. 17 and FIG. 18:
1700 a charger solution
1710 a charger
1712 a center part of the charger
1714 the surface of the charger on which the ring to be placed
1715 the other area of surface of the charger on which the ring to be placed
1716 the beveled part of the center of the charger
1718 a magnet (inside the base of the charger)
1720 a ring
1722 the bevel of the ring
1719 a charging coil of the charger
1723 a charging coil of the ring
1725 a vibrating element of the ring
1730 a power connector and cable (not shown) to the charger

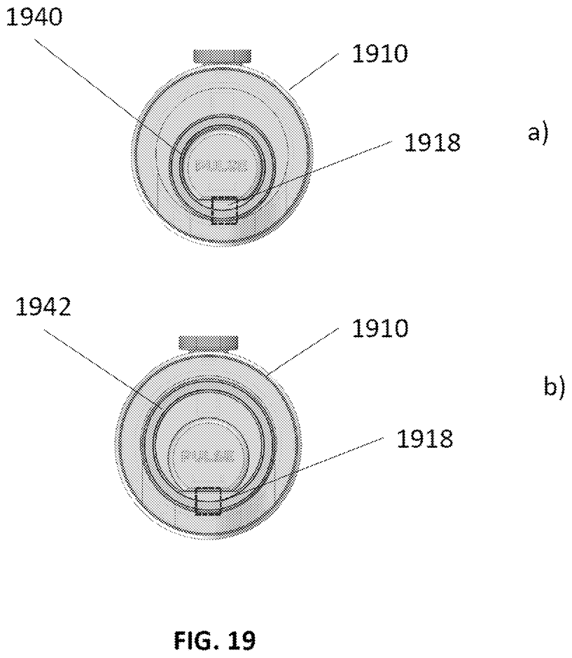

FIG. 19 and FIG. 20 and FIG. 21:
1910 a charger
1940 a ring, size 1
1942 a ring, size 2
1918 a magnet (inside the base of the charger)
1920 a first magnet (inside the base of the charger)
1922 a second magnet (inside the base of the charger)
1924, 1926 additional first magnets
1928, 1930 additional second magnets

FIG. 22:
2210 a charger
2220 a ring
2212 a center part of the charger
2214 the surface of the charger
2220 a first magnet (inside the base of the charger, North-pole shown on the top)
2240 a second magnet (inside the base of the charger, South-pole shown on the top)

### DESCRIPTION OF THE INVENTION

Small wearable devices, such as rings, typically lack displays or physical control buttons due to their compact size. This limitation poses challenges for implementing effective user interfaces, and therefore new interaction methods are needed for enabling command input, selections, and user feedback.

The present invention provides a solution based on tapping interactions. The wearable ring comprises a motion sensor capable of detecting motion caused by user actions, such as tapping the ring with another hand or finger, or by tapping the ring against a surface such as a table or wall. The motion sensor may include, for example, a 1 to 3-axis accelerometer or a piezoelectric sensor. These sensors can detect single or multiple sharp pulses corresponding to user-generated taps.

The detected pulses can be analyzed by the processor to extract distinguishing features such as frequency, intensity, duration, or sequence. The processor can be the processor of the ring device or the processor is embedded in the motion sensor. Examples of interpretable patterns include:
a short and strong single tap,
two successive light taps with a short pause,
a sequence of strong, medium, and light taps,
or a rhythm consisting of two short taps, a pause, and another two taps. It is understood that there are numerous pulse sequency, timing and delay variations. It is also possible that different letters are coded based for example as morse characters using dot and dash so that a dot has one pulse format and dash has another pulse format. Also certain words and sentences can be code in the same way.

The device allows for the creation, storage, and customization of such patterns. Users may define their own gesture codes and combinations, enabling the system to recognize a wide variety of tapping styles.

Once a valid pattern has been detected and interpreted, it may be stored in memory and represented either as raw signal data, time-frequency parameters, or as symbolic sequences such as “pulse-pause-double pulse-strong pulse.”

The device may include a wireless communication interface for transmitting the recognized pattern to a second device. The recipient device may be another ring, a smartwatch, or a smartphone. The second device may be equipped with a vibration element to reproduce the received pattern as haptic feedback.

In this manner, the invention enables direct tactile communication between two users. A user may send a tapping pattern to another user, who in turn can receive it as a haptic signal and optionally respond by repeating or modifying the pattern. The system thus supports interactive, low-latency communication between wearables.

Furthermore, the motion sensor may also be configured to detect the internal vibrations generated by the device's own vibration element. This enables precise synchronization of feedback and recognition logic. In addition, when two rings are in physical contact or close proximity, a second ring may detect vibrations generated by a first ring, further extending the range of inter-device interaction possibilities.

The disclosed system comprises a wearable ring device worn on a user's finger. The ring may also include a photoplethysmography (PPG) sensor configured to measure physiological signals including heart rate and heart rate variability. A motion sensor integrated into the ring detects physical movement and tapping gestures performed by the user.

The ring also comprises a vibration module capable of generating distinct haptic feedback signals. A controller and memory unit manage the operation of sensors, data collection, and communication tasks. A wireless communication module transmits sensor data to a paired mobile device over a short-range connection such as Bluetooth.

The mobile device comprises a processor that receives and analyzes physiological data from the ring to determine the user's current stress level. The mobile device maintains a record of historical stress levels and compares them with current values to generate context-aware feedback instructions.

Based on this analysis, the mobile device transmits vibration control data back to the ring, which activates the vibration module to provide feedback to the user. The feedback may indicate normal stress levels or recommend calming actions in cases of elevated stress.

The system further detects tapping feedback from the user as a response to the vibration cue. The mobile device evaluates this input and monitors subsequent physiological changes to determine the effectiveness of the guidance. The system may adapt future instructions accordingly.

In some embodiments, a second user may be equipped with a similar system comprising a second ring and mobile device. The two systems may communicate via a mobile network or internet-based service. Tapping gestures made by the first user may result in vibration feedback delivered to the second user's ring, enabling a novel, non-verbal form of communication.

The system may optionally include a web-based service or cloud infrastructure for long-term data storage, advanced analytics, or synchronized multi-user interaction.

### PREFERRED EMBODIMENTS

In a preferred embodiment, the wearable device is implemented as a ring designed to be worn on a human finger. The ring houses a three-axis accelerometer as the motion sensor, a compact linear resonant actuator (LRA) or eccentric rotating mass (ERM) motor as the vibration element, a microcontroller unit (MCU) for signal processing, and a Bluetooth Low Energy (BLE) module for wireless communication.

The motion sensor is configured to detect sudden changes in acceleration characteristic of user taps. The firmware includes a filtering algorithm to distinguish deliberate tapping gestures from background noise and motion artifacts. Once a pulse pattern is recognized, the processor classifies it using a rule-based or machine-learning model that maps features such as tap count, inter-tap delay, and signal magnitude to predefined commands.

The memory stores both factory-defined and user-defined tapping patterns. The user may enter a training mode through a long press or specific gesture, during which new tap sequences can be recorded and assigned to actions, such as:
triggering a silent notification,
sending a “ping” to another user,
initiating a mobile app function (e.g., answering a call or launching a smart home shortcut).

In another embodiment, the wearable device comprises a motion sensor to detect motion caused by tapping or contact with another object, a vibrating element to generate haptic signals, a processor, and a memory. The processor is configured to (i) analyze motion signals to identify a pattern of pulses based on at least one of frequency, intensity, and timing, and (ii) recognize motion signals resulting from the operation of the vibration element. The memory is configured to store said pattern or predetermined patterns or tapping or vibration parameters and instructions. (claim 1)

In another configuration the device comprises a communication interface configured to transmit the identified pattern to a second device. (claim 2)

In another configuration the processor of the device is further configured to categorize the identified pattern into predefined pattern types or encode it into symbolic representations. (claim 3)

In another configuration the second device comprises a wearable device or a mobile device. (claim 4)

In another configuration the second device comprises a vibration element configured to reproduce the transmitted pulse pattern as haptic feedback. (claim 5)

In another configuration the memory of the wearable device is further configured to store user-defined tapping or vibration instructions for responding to detected patterns. (claim 6)

In another configuration the second wearable device is configured to detect vibrations generated by the first device when the two devices are in contact or in close proximity. (claim 7)

In another configuration the processor is configured to compare the detected pattern to a library of user-defined patterns to trigger corresponding functions or commands. (claim 8)

In another configuration the device is configured to operate in a training mode for recording and labeling new tapping or vibration patterns defined by the user. (claim 9)

In another configuration the communication interface uses short-range wireless communication, including at least one of Bluetooth, ultra-wideband (UWB), or near-field communication (NFC). (claim 10)

In another configuration the device comprises a power management module configured to activate the motion sensor only when predefined movement conditions or wake gestures are detected. (claim 11)

In another configuration the vibration element is configured to generate modulated haptic feedback patterns corresponding to the received message or pattern classification. (claim 12)

In another configuration the second device is configured to forward received patterns to a cloud service or external system for processing, logging, or remote communication. (claim 13)

In another configuration the motion sensor is configured to detect additional gesture types including sliding or rotating movements of the ring. (claim 14)

In another configuration the device is integrated into a ring-shaped housing suitable for wearing on a human finger. (claim 15)

In further configuration the invention comprises a method in which a wearable device is used for detecting and transmitting a user interaction pattern. Motion caused by tapping or contact with another object is detected by a motion sensor of the wearable device. The vibration element is generating haptic signals in the wearable device. The processor analyzes the detected motion signals to identify a pattern of pulses based on at least one of frequency, intensity, and timing. Further the processor recognizes motion signals resulting from the operation of the vibration element; and stores the identified pattern or predetermined patterns or tapping or vibration parameters and instructions in a memory. (claim 16)

In further configuration the invention comprises the method which comprises transmitting the identified pattern to a second device via a communication interface. (claim 17)

In further configuration the invention comprises the method which comprises detecting, by a second wearable device, vibrations generated by the wearable device when the two devices are in contact or in close proximity. (claim 18)

In another embodiment, two rings worn by different users may communicate directly via BLE or indirectly via their paired smartphones and a cloud service. A tap pattern created by the first ring is transmitted wirelessly and reproduced as vibration on the second ring. The second user may respond with a mirrored pattern or send a different one, enabling a discreet two-way tactile communication channel.

In a further embodiment, the vibration element may emit haptic sequences that correspond to different patterns, allowing users to distinguish between messages based on the feedback alone. For instance, a strong-weak-strong vibration may correspond to an urgent alert, while a soft-soft-pause-soft sequence may indicate a casual message.

In yet another embodiment, a ring may be used in proximity or contact with another ring. The motion sensor of the second ring detects the vibration transmitted physically through the first ring, eliminating the need for radio communication in some interaction contexts (e.g., touch confirmation between partners).

The system may also include energy-saving features, such as wake-on-motion activation or proximity sensing using capacitive or IR-based detectors. These ensure the device remains dormant during inactivity and responsive during intended use.

### DETAILED DESCRIPTION

The present invention will now be described in detail with reference to the drawings and preferred embodiments. These examples are illustrative and not intended to limit the scope of the invention.

FIG. 1 illustrates a system architecture comprising a first user wearing a first ring (104a) and a second user wearing a second ring (104b). The first ring is wirelessly connected to a first mobile device via a short-range radio interface, such as Bluetooth, Zigbee or similar. The first mobile device is further connected to a mobile network, such as a cellular network or an internet-based web service. Similarly, the second ring is connected to a second mobile device via short-range radio, and the second mobile device is also connected to a mobile network or web service. Through this architecture, the first and second rings can communicate wirelessly either directly via short-range radio or indirectly via mobile network infrastructure or internet-based services. Further the first and second device can communicate with tapping-based interaction and communication using integrated motion sensing and vibration technology.

FIG. 2 and FIG. 3 show an example of a charging system for the ring. The ring is placed into a charging cradle that includes electrical contacts or wireless charging coils. The charger can be powered by a DC voltage source such as 5V for example through from USB cable or another battery operated voltage source.

FIG. 4 shows the internal modules of the ring device, including a motion sensor (420), a vibration module (422), a temperature sensor (424), an EDA (electrodermal activity) sensor (426), a controller (410), memory (412), and a communication module (414). In the preferred embodiments, the EDA and temperature sensors may be optional or selectively activated.

The components are powered by a battery through the power module. The sensors are operated and controlled by the processor. The processor can switch the sensors on and off and do the measurements continuously or time-to-time. The processor also controls the vibration module to cause a vibration element to vibrate. The processor can control the vibration frequency, amplitude/intensity and timing so that the processor can drive different vibration patterns which are recognizable by the user when the user is using a ring in his/her finger. Th temperature sensor can measure the temperature of the ring. The processor can analyze the temperature measured and judge if the ring is used in a finger or it is set on the charger. For example, if temperature is above 30 (Celsius) degrees the ring is most probably used in a finger. If the temperature is close to 20 (Celsius) degrees, it may be set to the charger. The motion sensor can be also used to determine if the ring is set to the charger. The motion is signal is very stable and only signal is close to 1 g when the ring is set to the charger. If the ring is used in a finger the acceleration signal typically varies and the acceleration signal can be detected in all 3 axes (x-, y- and z-) of the motion sensor.

FIG. 5. shows an example of the charger (500), including a controller (510), memory (512), communication module (514), battery (516), and power module (518). The charger may be powered by a known USB module or cable or having a rechargeable battery to charge the wearable device set on the charger. The controller, memory and communication modules can be optional or selectively activated depending on the construction of the charger.

FIG. 6A and FIG. 6B illustrate accelerometer data corresponding to tapping actions on the ring. The system identifies discrete pulses from raw motion signals to determine the user's tapping pattern and respond accordingly. Tapping action made by the user, the user taps the ring by another hand or finger, or the user touches another object, for example a table by a ring or hand, causes an acceleration signal in the output of the motion sensor. The motion sensor can be one axis, two-axis or three-axis. The signal from different axis can be monitored separately or the signals can be combined, for example summing each axis signal as squares. The processor can analyze the signal based on its amplitude or intensity or frequency or its timing. FIG. 6A shows one example of signal analysis. The acceleration signal (610) is compared to different threshold levels which may be set such as 0,1 g, 0,2 g or 0,5 g for example (the thresholds can be between 0,1-2 g). The thresholds can be also adaptive related to signal level detected during the last hour, 10 minutes or so. FIG. 6B shows that when the acceleration signal is above a threshold (630) but less than the threshold (620) the processor judges that there is a tapping pulse with a middle amplitude (650) detected at time t1. When the acceleration signal is above a threshold (620) the processor judges that there is a tapping pulse with a high amplitude (660) detected at time t2. When the acceleration signal is above a threshold (640) but less than the threshold (630) the processor judges that there is a tapping pulse with a high amplitude (670) detected at time t3.

FIGS. 7A, 7B, and 7C present various vibration signal patterns used to convey different feedback messages to the user, such as calming instructions or notifications. FIG. 7A shows three different vibration amplitudes at different times t1, t2 and t3. FIG. 7B shows three different vibration amplitudes at different times t1, t2 and t3. It is noted that delay (766) between the pulses 760 and 762 is different to the delay (768) between pulses 762 and 764. FIG. 7C shows three similar sequences of pulses which are repeated. The pulse pairs 770-772, 774-776, 778-780 are similar but they are repeated at different time points

FIGS. 8A, 8B, and 8C show how vibration patterns translate into motion signals inside the ring and how these can be interpreted by the system to verify that a vibration was received.

The motion sensor also detects the vibration element activity in a ring. FIG. 8B shows the acceleration signal detected by the motion sensor when the vibration element is driven at different amplitudes and frequencies (FIG. 8A). The processor can analyze the motion sensor signal and determine the pulse forms detected as shown in FIG. 8C. Pulses 840 and 842 are pulses representing higher frequency vibration with high amplitude. Pulses 844 and 846 are pulses representing lower frequency vibration with high amplitude. Pulse 848 represents pulse (808) at higher frequency vibration with small amplitude, pulse 850 represents pulse (810) at lower frequency vibration with small amplitude. The time stamps of the pulses 840-850 relates to the original vibration pulses 800-810), so that timing, duration and delays between the pulses can be recognized.

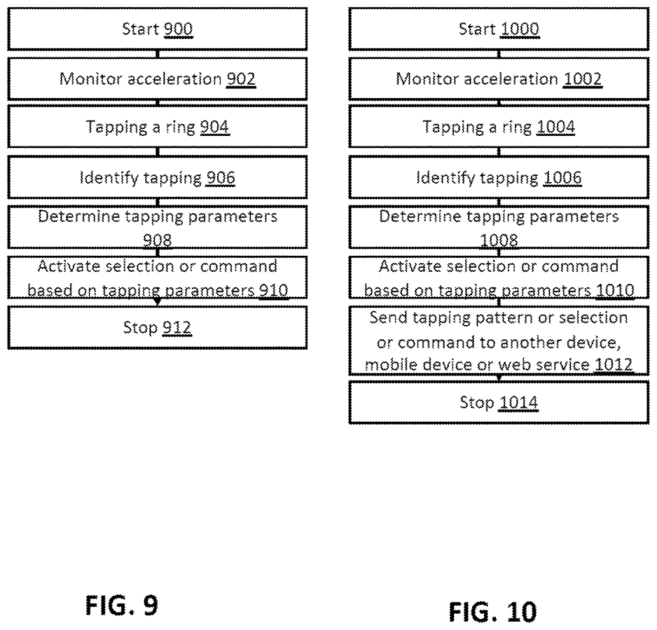

FIGS. 9 and 10 depict tapping-based user input. The user taps the ring to confirm receipt of a vibration cue or initiate communication. The motion sensor detects and the processor classifies these gestures to trigger system actions.

First the process starts at (900,1000). The processor starts with the motion sensor to detect acceleration of a ring (902, 1002). The user taps the ring with a selected pattern, for example once, two times, or two higher amplitude and one lower amplitude (904, 1004). The motion sensor detects the acceleration signal caused by tapping (906, 1006). The processor analyses and determines the tapping parameters such as intensity, frequency, timing and delay (908, 1008). The processor can activate selection (910, 1010), for example to save the tapping pattern, initiate sending the tapping sequency or tapping parameters or a message related to tapping parameters by a short-range radio link (e.g. bluetooth) to a mobile device or web service to another short-range radio device (1012). The message can be, for example, thank you, hello, ok, I love you, See you soon. The message can be based on tapping parameters and predetermined rules or mutual agreement between users how the letters, words or messages has been coded into different pulses (amplitudes, delays, timings and number of pulses).

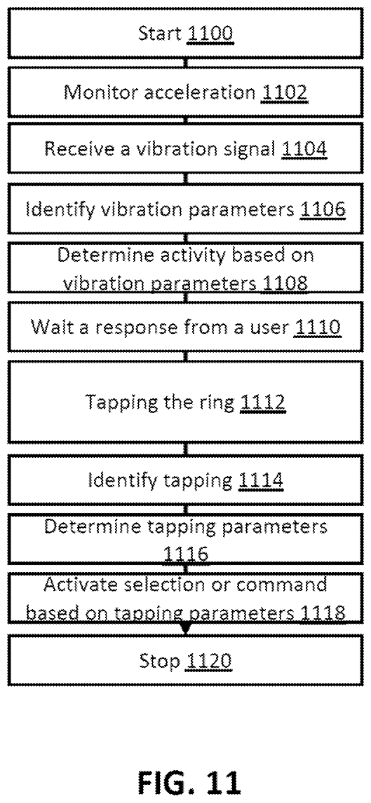

FIG. 11 demonstrates a combined use case where the users communicate through two ring devices both having vibration feedback and tapping input form a closed-loop feedback interaction. The process can be as follows: It starts at 1100. The processor starts the motion sensor to monitor acceleration (1102). The device receives a vibration signal. This may be message received through a short-range radio from another device or a mobile device or a web service. The message may be coded or named as “thank you, hello, ok, I love you, See you soon, relax, take easy” or just being pulse sequency. The message is then played by the vibration element driven by the processor in the device. The vibration is then received and detected by a motion sensor. The vibration parameters are then analyzed and identified, such as frequency, intensity, timing and/or delays. The activity is then determined based on the vibration parameters. The determination can be for example wait a response, repeat the message etc. The user may also determine the activity to make the response to vibration message. The device will wait then the response from the user (1110). If user taps the device (1112) the device identifies tapping by measuring tapping motion by the motion sensor (1114). determine tapping parameters and activate selection or command as described above and FIG. 9 and FIG. 10.

FIG. 12 demonstrates a combined use case where both vibration feedback and tapping input form a closed-loop feedback interaction.

FIG. 12 shows how the two ring devices can be paired or change the messages. Both devices will be started (1200, 1216) and set to monitor acceleration signals (1202, 1218). The ring 1 is moved in a close contact or touch to the ring 2 or wise versa, so that vibration signal from one ring can be transferred to another device. The ring is set to send a vibration signal (1206) the ring 2 will receive the signal from the ring 1 (1220). The motion sensor of ring 2 will detect the vibration signal as an acceleration signal and the processor of the ring 2 will identify the vibration parameters (1222). The processor of the ring 2 device will determine the activity based on the vibration parameters (1224). The activity can be for example “wait a response from a user 2” (1226), “create vibration feedback” (1238), or “record and save the vibration pattern or vibration parameters” (1240). The saved vibration pattern or vibration is now recorded for further use and recognition. The ring 2 will recognize the same pattern later and identify that it is the same pattern as received from ring 1. So, the received message having the same pattern is then associated with the ring 1 and its user, for example. The pattern can be for example as described in FIG. 8C comprising two short and intensive pulses (840, 842) followed by a longer and lower intensity pulse (844), the pulses may be driven with the delays of 0.5 second between pulses 840 and 842 and the delay of 0.6 seconds between the pulses 842 and 844. Any other combinations with different delays between the pulses can be used. The intensities of the pulses can be varied as a part of the pattern and code. Typically, the number of the pulses in one pattern can be 2-5,but it can be also more than 5 or more than 10. The ring 2 can also create vibration feedback to be sent or transferred to the ring 1. The feedback signal can be the same pattern as received to ensuring the the ring 2 has received the vibration signal, or it can be unique pulse pattern related to the ring 2 and user 2.

In the case the ring 2 waits for the response from the user 2, the user 2 will tap the ring 2. The ring 2 can identify the tapping as described in steps 902-912 as shown in FIG. 9. If the rings are still close to each other so that tapping (1228) of the ring 2 will cause motion signal in the ring 1 or vibration signal cause a motion signal in the ring 1 (1238), the ring 1 will receive the tapping or vibration signal feedback from the ring 2. The ring 1 can identify the tapping as described in steps 902-912 as shown in FIG. 9.

### A Stress Monitoring Device and Instruction Method Based on a Vibration Element

Smart rings present a challenge due to their compact size and lack of user interface components such as screens or physical buttons. This makes it difficult to provide meaningful, real-time feedback or to receive user input directly through the device.

Existing systems may offer alerts via mobile applications or vibration, but they often lack a closed-loop feedback system that evaluates user responses based on physiological changes. Furthermore, discreet and intuitive communication methods between the user and the device—or between multiple users—remain largely undeveloped.

The invention provides a system comprising a wearable ring device and a mobile device configured for bi-directional communication. The ring includes a PPG sensor for measuring physiological signals such as heart rate and heart rate variability, and a motion sensor for detecting tapping gestures. The mobile device receives the data, determines the user's stress level, and activates a vibration module in the ring to provide user feedback based on the stress analysis. The stress analysis can be based, for example, on the heart rate level, heart rate variability and their combination. Different methods are for example discussed in the scientific paper: Kim et al. Stress and Heart Rate Variablity (2018) (https://pmc.ncbi.nlm.nih.gov/articles/PMC5900369/)

Importantly, the invention addresses the inherent user interface limitations of ring-type wearable devices, which typically lack displays or physical buttons. By leveraging motion sensing and haptic feedback, the system enables intuitive, screenless interaction with the user. This allows the ring to provide meaningful guidance and receive confirmation or feedback through simple tapping gestures.

The system further detects user interaction via tapping, evaluates whether the haptic instruction led to a physiological response (e.g., stress reduction), and adapts future feedback accordingly. The invention may also support communication between multiple users via tapping and vibration, enabling a silent and intuitive form of interaction.

These following embodiments illustrate a non-intrusive and display-free user interface that leverages physiological sensing, vibration feedback, and motion-based input to enable continuous, interactive stress monitoring and communication.

FIG. 1 illustrates an example of a system comprising a first user wearing a first ring and a second user wearing a second ring. The first ring is wirelessly connected to a first mobile device via a short-range radio interface, such as Bluetooth. The first mobile device is further connected to a mobile network, such as a cellular network or an internet-based web service. Similarly, the second ring is connected to a second mobile device via short-range radio, and the second mobile device is also connected to a mobile network or web service. Through this architecture, the first and second rings can communicate wirelessly either directly via short-range radio or indirectly via mobile network infrastructure or internet-based services. Further the first and second device can communicate with tapping-based interaction and communication using integrated motion sensing and vibration technology.

FIG. 13 illustrates a system comprising a ring, a mobile device and web service, and their elements and modules

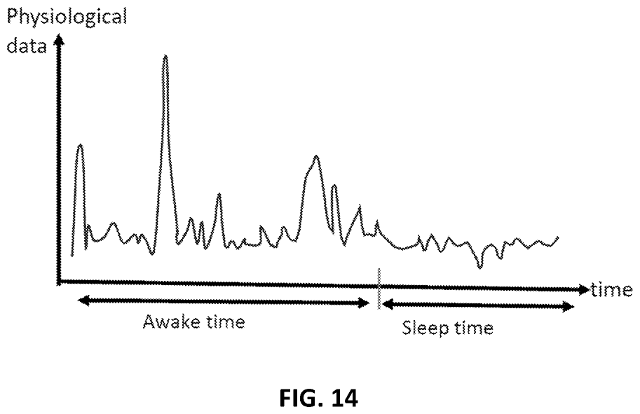

FIG. 14 illustrates an example of physiological signal measured by a ring showing measurement signal during awake time and sleep time.

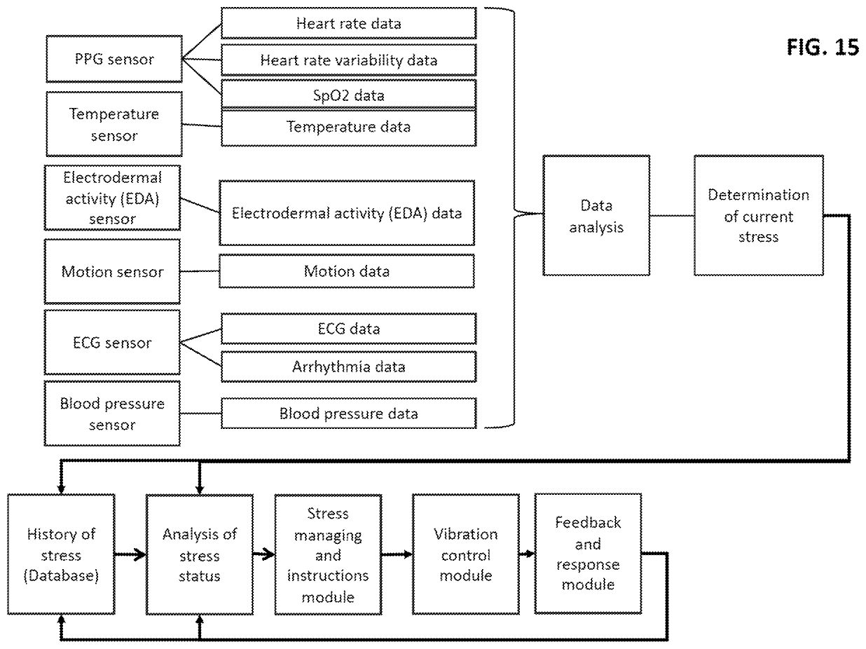

FIG. 15 illustrates a high-level flow of the system operation: sensor data collection, data analysis, stress status determination, and feedback via vibration

FIG. 16 provides a detailed flowchart of the stress management logic

The disclosed system comprises a wearable ring device worn on a user's finger. The ring includes a photoplethysmography (PPG) sensor configured to measure physiological signals including heart rate and heart rate variability. A motion sensor integrated into the ring detects physical movement and tapping gestures performed by the user.

The ring also comprises a vibration module capable of generating distinct haptic feedback signals. A controller and memory unit manage the operation of sensors, data collection, and communication tasks. A wireless communication module transmits sensor data to a paired mobile device over a short-range connection such as Bluetooth.

The mobile device comprises a processor that receives and analyzes physiological data from the ring to determine the user's current stress level. The mobile device maintains a record of historical stress levels and compares them with current values to generate context-aware feedback instructions.

Based on this analysis, the mobile device transmits vibration control data back to the ring, which activates the vibration module to provide feedback to the user. The feedback may indicate normal stress levels or recommend calming actions in cases of elevated stress.

The system further detects tapping feedback from the user as a response to the vibration cue. The mobile device evaluates this input and monitors subsequent physiological changes to determine the effectiveness of the guidance. The system may adapt future instructions accordingly.

In some embodiments, a second user may be equipped with a similar system comprising a second ring and mobile device. The two systems may communicate via a mobile network or internet-based service. Tapping gestures made by the first user may result in vibration feedback delivered to the second user's ring, enabling a novel, non-verbal form of communication.

The system may optionally include a web-based service or cloud infrastructure for long-term data storage, advanced analytics, or synchronized multi-user interaction.

Preferred embodiments of the invention are described with reference to the accompanying drawings.

FIG. 13 provides a block diagram of the complete system including the ring (104), mobile device (106), and optional web service (108). The ring collects physiological data and transmits it to the mobile device, which processes it via a data processing module (1354). Stress level determination and instruction generation are handled locally. The mobile device may also send summary data to the web service, where historical data is stored in a database (1356 and or 1396) and used for trend analysis. FIG. 13 shows the internal modules of the ring device, including a motion sensor (1320), a vibration module (1322), a temperature sensor (1324), an EDA (electrodermal activity) sensor (1330), a PPG module (1326), an ECG module (1328), a controller (410), memory (412), and a communication module (414). In the preferred embodiments, the EDA sensor, ECG module and temperature sensor may be optional or selectively activated.

FIG. 14 shows physiological signals recorded over time, highlighting variations during awake and sleep states. These variations may be used as context in stress-level analysis. Awake and sleep status can be determined based on time of the day or based on known method to determine sleep status and stages based on the measured PPG signal (heart rate) and temperature or other sensor signals.

FIG. 15 illustrates a high-level flow of the system operation: sensor data collection, data analysis, stress status determination, and feedback via vibration. Stress status determination van be based on current method to determines stress level of a user based on low level of heart rate variability, higher level of heart rate, changes in EDA signal or temperature signal. The system monitors physiological signals, determines stress level, delivers haptic feedback, verifies receipt via tapping, and evaluates the effectiveness of the guidance through follow-up measurements.

FIGS. 16A, 10B, and 10C present various vibration signal patterns used to convey different feedback messages to the user, such as calming instructions or notifications.

FIGS. 11A to 11C show how vibration patterns translate into motion signals inside the ring and how these can be interpreted by the system to verify that a vibration was received.

FIGS. 12 and 13 depict tapping-based user input. The user taps the ring to confirm receipt of a vibration cue or initiate communication. The motion sensor detects and classifies these gestures to trigger system actions.

FIG. 14 demonstrates a combined use case where both vibration feedback and tapping input form a closed-loop feedback interaction.

FIG. 15 provides a detailed flowchart of the stress management logic. The system monitors physiological signals, determines stress level, delivers haptic feedback, verifies receipt via tapping, and evaluates the effectiveness of the guidance through follow-up measurements.

FIG. 16 provides a detailed flowchart of the stress management logic. The process comprises starting the system and monitoring (1612). The system uses the sensor in a ring to monitor the user (1614). The system uses different sensors to collect data (1616). Then the system analyses the data (1618) to evaluate trends changes and status to determine the current stress level of the user (1620). The system will use also historical data for comparing it to current level and analysing the user base level to determine normal level of stress or variation of the stress level based on data/signals levels of the sensors or their combinations (1622). Next the system will evaluate different options to provide guidance and instructions to the user (1624). The indication can be done using a vibration element or also using the display of a mobile device or a computer display connected to the ring device. The system will provide vibrational indication to the user through the vibration element in the ring (1626). The motion sensor of the ring will also detect the vibration signal provided (1628). The motion sensor is also detecting the tapping of the ring generated by the user (1630). The sensor identifies the motion of the ring can determine if it caused by the vibration element or by tapping by the user. Based on the timing between the vibration signal and tapping, the processor can determine if the user has received the vibration signal and thus stress management guidance (1632). The delay between vibration signal and tapping may be for example 0.5-5 seconds. The system can further monitor the stress level so that the processor can determine if the instruction and guidance for stress management through the vibration has been effective, and how the stress level has been changed after the guidance (1634).

These embodiments illustrate a non-intrusive and display-free user interface that leverages physiological sensing, vibration feedback, and motion-based input to enable continuous, interactive stress monitoring and communication.

In one embodiment a system for monitoring and managing the stress level of a user, comprises a wearable ring device, which comprises one or more physiological sensors including a photoplethysmography (PPG) sensor configured to detect at least heart rate and heart rate variability, a motion sensor configured to detect motion data and tapping gestures, a vibration module configured to generate haptic feedback, a controller and memory configured to operate said sensors and module, a communication module configured to transmit and receive data wirelessly to and from an external device; and mobile device which comprises a communication interface configured to receive physiological and motion data from the ring device, a processing module configured to analyze received sensor data to determine a current stress level of the user, compare the current stress level with historical data stored in a database, generate vibration control data and instruction data based on said analysis, a transmission module configured to send said control and instruction data back to the ring device. The vibration module in the ring device is activated to provide stress-related feedback or instructions to the user based on the determined stress level. The instructions may include at least: an indication that stress level is within normal range or an instruction to take action to reduce elevated stress. The system is further configured to detect user response via tapping gestures on the ring as sensed by the motion sensor, evaluate, based on subsequent physiological data, whether the vibration feedback resulted in a change in the user's stress level, update stress management instructions accordingly based on said evaluation.

In another embodiment the motion sensor of the system is further configured to detect a predefined tapping pattern as user feedback.

In another embodiment the vibration module of the system is configured to produce different vibration patterns corresponding to different stress levels or instructions.

In another embodiment the processing module of the mobile device uses a machine learning algorithm to determine the stress level based on physiological sensor data and historical trends.

In another embodiment the historical stress data is stored locally in the memory of the mobile device.

In another embodiment the user feedback detected via tapping is used to confirm the receipt of the vibration instruction.

In another embodiment the system delays subsequent vibration feedback until a change in the user's physiological data is detected following a prior instruction.

In another embodiment the vibration module is operable at varying intensities and durations to convey different types of guidance or urgency levels.

In another embodiment the ring device includes a rechargeable battery and a charging interface compatible with a docking charger.

In another embodiment the mobile device transmits a summary of stress level and feedback interactions to an external application or service for record-keeping or analytics.

In another embodiment the system comprises a second wearable ring device worn by a second user and a second mobile device, wherein the first and second mobile devices are configured to communicate over a mobile or internet network.

In another embodiment a tapping gesture detected on the first ring device by the first user is transmitted via the first mobile device to the second ring device, and a corresponding vibration is triggered on the second ring device.

In another embodiment the second ring device detects the vibration, and the second user provides a tapping response, which is transmitted back to the first user as a reciprocal interaction.

In another embodiment the mobile devices and ring devices are configured to support bi-directional communication of tapping and vibration signals as a form of silent or non-verbal interaction.

In another embodiment the tapping patterns can be predefined to correspond to specific messages or emotional states shared between users.

### A Charger of a Wearable Device

In one aspect, the invention provides a wireless charging apparatus comprising: a surface for positioning the ring-shaped wearable device during charging; a charging coil embedded within the apparatus; a central region associated with the alignment of the ring; a first magnet located beneath the charging surface to attract the ring to a first predefined position, aligning the ring against the surface for optimal charging.

In other aspects: a second magnet may be positioned to attract a second location on the ring (C2).

In another aspect: two or more magnets may be positioned along the circumference beneath the charging surface to follow the ring's shape and ensure radial alignment (C3).

In another aspect: the magnets may be radially symmetrical (C4).

In another aspect: the first magnet may attract a magnet or magnetic part located within the vibrating element of the ring (C5).

In another aspect: alternatively, the magnets may attract the magnetic material of the ring body itself (C6).

In another aspect: the magnets may be of different shapes, e.g., cylindrical for the first magnet and rectangular for the second (C7).

In another aspect: the thickness of the charging surface may be reduced in the region where the ring is placed, e.g., 0.1-3 mm thinner than the surrounding areas, to improve magnetic coupling (C8).

In another aspect: the charging surface material may differ in hardness or softness relative to the rest of the apparatus (C9).

In another aspect: suitable materials for the surface include stainless steel, aluminum, or titanium (C10).

In another aspect: the surface may be configured to generate an audible indication (e.g., click, snap, or tone) when the ring contacts it (C11).

In another aspect: the magnets may be polarized with their north and south poles horizontally aligned on top of each other (C12).

In another embodiment, the first and second magnets may be oriented with different horizontal polarities, to guide different parts of the ring into specific alignment (C13).

FIG. 2 and FIG. 3 illustrate a wireless charging solution (200,300) comprising a charger (210,310) and a ring-shaped wearable device (320). The charger (210,310) includes a center part (212,312), a surface (214,314) on which the ring (320) is placed, and a beveled portion (216) in the center region. The ring (320) includes a corresponding beveled inner surface (322) designed to align with the beveled portion (216) of the charger for proper positioning during charging.

FIG. 17 and FIG. 18 show a more detailed wireless charging apparatus (1700) comprising a charger (1710) and a ring (1720). The charger includes a center part (1712), a charging surface (1714) for receiving the ring, and an adjacent surface area (1715) surrounding the center.

### A Beveled Part (1716) in the Center Facilitates Alignment With the Ring's Inner Bevel (1722).

A magnet (1718) is embedded within the charger base to aid magnetic positioning.

The charger also includes a charging coil (1719), while the ring houses a corresponding receiving coil (1723) and a vibrating element (1725). A power connector and cable (1730, not shown) are provided to supply power to the charger. The charger can be powered by a USB cable or similar. The charger may comprise a controller and charging circuitry for providing the wireless power through charging antenna or charging coil to the ring to be charged.

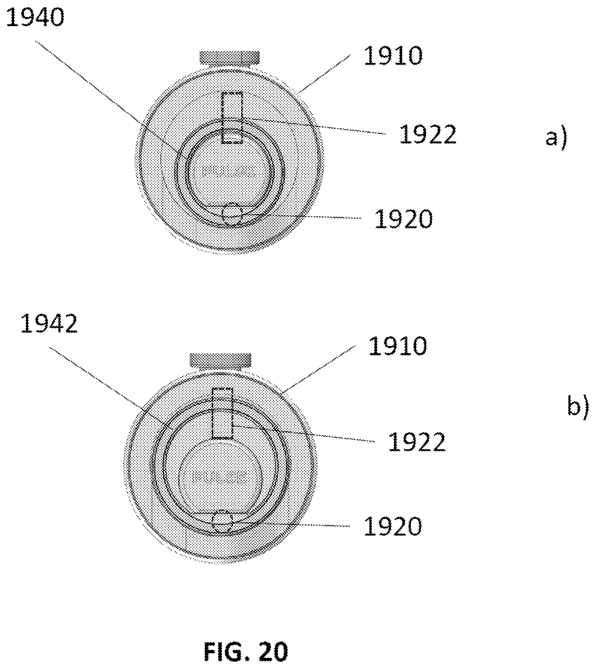

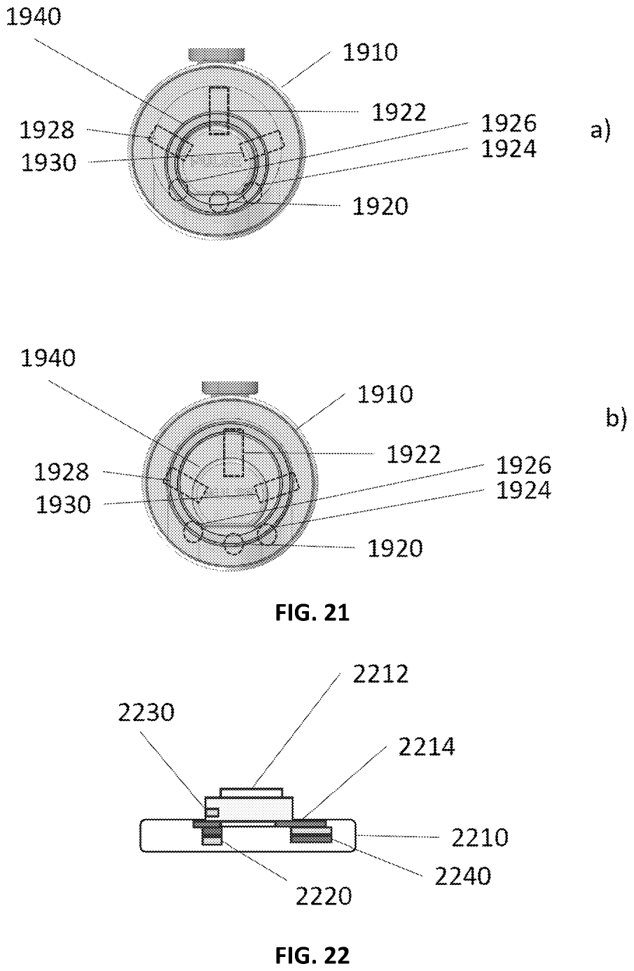

FIG. 19, FIG. 20, and FIG. 21 depict different ring sizes and magnet arrangements. The charger (1910) is shown with two ring sizes: a smaller ring (1940) and a larger ring (1942).

Magnets are embedded in the charger base to facilitate alignment and secure placement: a general magnet (1918), a first magnet (1920), and a second magnet (1922).

Additional first magnets (1924, 1926) and second magnets (1928, 1930) may be positioned along the ring's circumference to enhance magnetic attraction and stability.

FIG. 22 illustrates another embodiment of the charger (2210) and ring (2230). The charger includes a center part (2212) and a surface (2214) for ring placement. A first magnet (2220) is positioned within the base of the charger, with the north pole facing upward, and a second magnet (2240) is also embedded with the south pole facing upward. This arrangement facilitates controlled magnetic guidance and alignment of the ring.

### Overview of the System

A wireless charging apparatus is designed for charging a ring-shaped wearable device. The apparatus includes a surface of the charger, which is the area on which the ring is placed during charging. The charger comprises a central part in which the charging coil is arranged within a beveled portion of the charger structure. Correspondingly, the charging coil of the ring is positioned inside a beveled portion of the ring itself, which is designed to match the bevel of the charger. These matching beveled portions ensure that the ring is always oriented in the correct radial and rotational position during charging. The charging coil of the charger is configured to wirelessly transmit power to the corresponding receiving coil located within the ring.

The apparatus further comprises at least one magnet positioned beneath the surface of the charger, which serves to attract the ring and align it with the correct charging position.

### Magnetic Alignment and Positioning

A first magnet is located beneath the surface of the charger, positioned to attract a corresponding magnetic portion of the ring and guide it into a first location on the surface. The magnetic portion of the ring is for example a magnet or magnetic part or vibration element such as a vibration motor. The magnet or magnetic part may be also a separate magnet or magnetic part of the ring such as material or ring or metallic portion of the ring cover or so. The magnetic part is optimally arranged to be on the beveled portion of the ring. This magnetic force pulls the ring flush against the surface of the charger, ensuring that the ring remains horizontally aligned and at a consistent vertical distance from the charging coil. This alignment is essential for efficient wireless energy transfer.

In some embodiments, a second magnet is also positioned beneath the surface of the charger to attract the ring at a second location, enhancing the overall stability and orientation of the ring on the charger.

In further embodiments, two or more magnets are positioned beneath the surface of the charger along a circular path that corresponds to the circumference of the ring. This arrangement helps ensure that the ring is radially and rotationally aligned with respect to the central region of the charger. The ring may comprise other magnets or magnetic parts along the inner or outer surface of the ring corresponding to the magnets positioned beneath the surface of the charger along the circular path corresponding to the circumference of the ring.

The magnets may be arranged radially symmetrically around the central axis to generate balanced magnetic forces that hold the ring evenly in place regardless of its size.

### Interaction with the Ring Device

The magnets of the charger may be configured to attract various elements of the ring, including:
a magnet or magnetic element integrated into a vibration module inside the ring; or
magnetic material forming part of the ring's structure.

The use of magnets allows the user to place the ring with minimal visual alignment, as the magnetic force will guide and secure the ring automatically into the correct charging position. This feature is especially beneficial in low-light conditions or when the user relies on tactile feedback.

### Feedback and User Experience

To improve user interaction, the surface of the charger may be designed to provide auditory feedback when the ring makes proper contact—such as a distinct sound (e.g., a snap or click). This confirms that the ring is correctly seated and held in place.

The surface of the charger may be thinner than the other surface area, which may be 0.7-2.0 mm thick, in the area where the ring rests, enhancing magnetic attraction and coil coupling. The surface area of receiving the ring may be e.g., by 0.1-0.7 mm, or 0.1-0.4 mm or 0.1-0.2 mm thinner than the other surface area.

The material of the surface may differ from the rest of the apparatus, being either softer or harder, depending on desired tactile or acoustic properties.

Suitable materials for the surface of the charger may include stainless steel, aluminum, or titanium for durability and responsiveness.

### Magnet Configurations

The first magnet beneath the surface of the charger may have north and south poles aligned horizontally on top of each other. In some embodiments, different magnets (e.g., the first and second) may have their poles aligned in different horizontal orientations, allowing them to exert directed forces that further assist with the ring's orientation and stability.

Alternative Embodiments in some implementations:

The magnets may differ in shape and strength—for example, a cylindrical first magnet and a rectangular second magnet.

The system may be compatible with rings of various sizes, as the magnetic and geometric features of the charger allow consistent positioning regardless of diameter.

1. A wearable device comprising:
a motion sensor configured to detect motion caused by tapping or contact with another object;
a vibration element configured to generate haptic signals;
a processor configured to:
(i) analyze motion signals to identify a pattern of pulses based on at least one of frequency, intensity, and timing, and
(ii) recognize motion signals resulting from the operation of the vibration element;
a memory configured to store said pattern or predetermined patterns or tapping or vibration parameters and instructions.

2. The device of claim 1, further comprising a communication interface configured to transmit the identified pattern to a second device.

3. The device of claim 1, wherein the processor is further configured to categorize the identified pattern into predefined pattern types or encode it into symbolic representations.

4. The device of claim 2, wherein the second device comprises a wearable device or a mobile device.

5. The device of claim 4, wherein the second device comprises a vibration element configured to reproduce the transmitted pulse pattern as haptic feedback.

6. The device of claim 1, wherein the memory is further configured to store user-defined tapping or vibration instructions for responding to detected patterns.

7. The device of claim 1, wherein a second wearable device is configured to detect vibrations generated by the first device when the two devices are in contact or in close proximity.

8. The device of claim 1, wherein the processor is configured to compare the detected pattern to a library of user-defined patterns to trigger corresponding functions or commands.

9. The device of claim 1, wherein the device is configured to operate in a training mode for recording and labeling new tapping or vibration patterns defined by the user.

10. The device of claim 2, wherein the communication interface uses short-range wireless communication, including at least one of Bluetooth, ultra-wideband (UWB), or near-field communication (NFC).

11. The device of claim 1, further comprising a power management module configured to activate the motion sensor only when predefined movement conditions or wake gestures are detected.

12. The device of claim 1, wherein the vibration element is configured to generate modulated haptic feedback patterns corresponding to the received message or pattern classification.

13. The device of claim 4, wherein the second device is configured to forward received patterns to a cloud service or external system for processing, logging, or remote communication.

14. The device of claim 1, wherein the motion sensor is configured to detect additional gesture types including sliding or rotating movements of the ring.

15. The device of claim 1, wherein the device is integrated into a ring-shaped housing suitable for wearing on a human finger.

16. A method for detecting and transmitting a user interaction pattern using a wearable device, comprising:
detecting, by a motion sensor of the wearable device, motion caused by tapping or contact with another object;
generating, by a vibration element, haptic signals in the wearable device;
analyzing, by a processor, the detected motion signals to identify a pattern of pulses based on at least one of frequency, intensity, and timing;
recognizing, by the processor, motion signals resulting from the operation of the vibration element; and
storing, in a memory, the identified pattern or predetermined patterns or tapping or vibration parameters and instructions.

17. The method of claim 16, further comprising transmitting the identified pattern to a second device via a communication interface.

18. The method of claim 16, further comprising detecting, by a second wearable device, vibrations generated by the wearable device when the two devices are in contact or in close proximity.
